(19) United States
(12) Patent Application Publication
(10) Pub. No.: US 2021/0338933 A1
(43) Pub. Date: Nov. 4, 2021

(54) AUTOINJECTION DEVICE HAVING DOSE LOGGING

(71) Applicant: Novo Nordisk A/S, Bagsvaerd (DK)

(72) Inventor: Nikolaj Eusebius Jakobsen, Soeborg (DK)

(21) Appl. No.: 17/261,730

(22) PCT Filed: Jul. 17, 2019
(86) PCT No.: PCT/EP2019/069274

§ 371 Date: Jan. 20, 2021

(30) Foreign Application Priority Data
Jul. 20, 2018 (EP) 18184848.2

(51) Int. Cl. A61M 5/20; A61M 5/315

(52) U.S. Cl. CPC A61M 5/2033; A61M 5/31578; A61M 2005/31508; A61M 5/31535; A61M 5/31501

(57) ABSTRACT

An autoinjection device (10′) for expelling a dose of drug is described. A housing (300) movably holds a power unit (400, 500, 550, 700) configured for driving the piston of a held container (100), the power unit comprising: a) a plunger (500) comprising a retaining geometry (515), b) a drive spring (550) arranged in a tensed state wherein a first end portion of the drive spring (550) provides a distally directed force on the plunger (500), and c) a power base (400) operably coupled to the drive spring (550) and the plunger (500), the power base (400) grounding a second end portion of the drive spring (550), wherein, in a pre-firing state of the autoinjector, a retaining element (410, 415) of the power base (400) releasably engages the retaining geometry (515) of the plunger to retain the plunger against the force of the drive spring (550), wherein the autoinjection device (10′) further defines a user operable trigger element (700) cooperating with the retaining element (410, 415) to maintain retaining engagement with the retaining geometry (515) of the plunger until triggering, and further defines an electronic module (80′) comprising a sensor (850, 851) configured to sense the shift of position of the power base (400) as it moves upon triggering.

### FIELD OF THE INVENTION

The present invention relates to injection devices for injecting a drug. In particular the present invention relates to an autoinjection device having a releasable plunger, and an electronic module for sensing when the plunger has been released for injecting the drug.

### BACKGROUND OF THE INVENTION

In relation to some diseases patients must inject a drug on a regular basis, such as once weekly, once daily or even a plurality of times each day. Drug injection devices have greatly improved the lives of patients who must self-administer drugs and biological agents. Drug injection devices may take many forms, including simple disposable devices that are little more than an ampoule with an injection means or they may be durable devices adapted to be used with replaceable pre-filled cartridges. Regardless of their form and type, they have proven to be great aids in assisting patients to self-administer injectable drugs and biological agents. They also greatly assist care givers in administering injectable medicines to those incapable of performing self-injections.

A particular class of drug injection devices gaining increasing interest is the so-called autoinjector, wherein a drug is automatically expelled from the device upon triggering of the expelling mechanism. Some autoinjectors are disposable offering a single or a multitude of individual dose deliveries, other are durable for use with an exchangeable drug container.

Performing the necessary injection of a drug at the right time, the right type, and sometimes, in the right size is essential for managing therapy of a disease, i.e. compliance with the specified drug injection regimen is important. In order to make it possible for medical personnel to determine the effectiveness of a prescribed dosage pattern, some patients, such as diabetes patients, are encouraged to keep a log of the size and time of each injection. However, such logs are normally kept in handwritten notebooks, from the logged information may not be easily uploaded to a computer for data processing. Furthermore, as only events, which are noted by the patient, are logged, the note book system requires that the patient remembers to log each injection, if the logged information is to have any value in the treatment of the patient's disease. A missing or erroneous record in the log results in a misleading picture of the injection history and thus a misleading basis for the medical personnel's decision making with respect to future medication. Accordingly, it may be desirable to automate the logging of ejection information from medication delivery systems. Thus, a number of injection devices with a dose monitoring/acquisition feature have been provided, see e.g. in WO 2017/129314 and WO 2018/085952.

WO 2017/129314 discloses an electrical information device in combination with an auto-injection device, wherein both a start of delivery sensor and an end of delivery sensor is provided. The end of delivery sensor senses movement of an end of dose signaling member which, when the medicament has been delivered, is released and driven towards the rear of the injector by a drive-spring. In order to sense the end of delivery condition, a dedicated end of dose signal member is thus required.

WO 2018/085952 discloses an autoinjection system wherein an electronic add-on module is to be fitted releasably onto a first autoinjector before the start of injection. After use, the add-on module is transferred to a second autoinjector for re-use of the add-on module with the new autoinjector. Although the cost for electronic components may be lowered by such system, when used with autoinjectors configured for single dose administration, the additional steps of fitting the add-on module adds to the complexity of use and may not be optimal from a user's point of view.

Disposable devices require production costs to be low, since they have a very limited lifetime. Furthermore, the environmental impact of implementing traditional electronic circuitry in disposable devices limits the possibilities of practical use of such logging devices in disposable devices. Thus, in order to provide a viable solution, simplicity of an electrically enabled injector is of major importance.

Having regard to the above-identified prior art devices, it is an object of the present invention to provide an autoinjector having dose logging which enables an inexpensive production setup, wherein only a minimum number of variants of components and/or assemblies are needed for creating different versions of the autoinjector.

A further object is to provide an autoinjection device that is improved having regard to monitoring the triggering of an autoinjector, and wherein the device is less sensitive to noise and to tolerance variations.

Yet additional further objects of the invention are to provide measures for obtaining an autoinjector having a superior performance and, at the same time, enabling manufacture with a minimum of components, and at a reduced cost.

### BRIEF DESCRIPTION OF THE INVENTION

In a first aspect the present invention relates to an autoinjection device for expelling a dose of drug, the injection device comprising:
a housing having a proximal end (P) and a distal end (D),
a drug container comprising a container barrel extending along an axis, a distal outlet connectable or connected to an injection needle, and a piston that is sealingly and slideably arranged inside the container barrel,
a power unit configured for driving the piston distally along the axis to expel a drug contained in the drug container, the power unit comprising:
   a plunger adapted for cooperation with the piston to drive the piston distally along a central axis, the plunger comprising a retaining geometry,
   a drive spring arranged in a tensed state wherein a first end portion of the drive spring acts on the plunger with a force biasing the plunger distally, and
   a power base operably coupled to the drive spring and the plunger, the power base grounding a second end portion of the drive spring, wherein, in a pre-firing state, a retaining element of the power base releasably engages the retaining geometry of the plunger to retain the plunger against the force of the drive spring,
a user operable trigger element cooperating with the retaining element and shiftable from a pre-firing state wherein the trigger element cooperates with the retaining element to maintain retaining engagement with the retaining geometry of the plunger, and into a firing state wherein said retaining engagement is released,

wherein the power base is movably arranged in the housing and configured to move proximally, due to a proximally directed force provided by the drive spring, from a first pre-firing position into a second fired position upon release of the retaining engagement, and an electronic module arranged relative to the housing, the electronic module comprising a sensor configured to sense the shift of position of the power base as it moves from the first pre-firing position into the second fired position.

By providing the power base movably arranged relative to the housing, this enables use of the power base as a delivery indicating member which allows the electronic module to monitor delivery of the drug directly by monitoring the position of the power base. Hence, a very simple solution is obtained.

The autoinjector may be provided in a form configured so that, when the trigger element assumes the pre-firing state, the power unit is arranged axially floating relative to the housing.

In some forms of the autoinjector the retaining element is unitarily formed with the power base. In some embodiments, the power base includes a proximal portion forming a cap shaped element.

In some embodiments the power unit further comprises said user operable trigger element.

In certain embodiments the retaining element defines an arm extending from a base section of the power base towards a plunger engagement portion, wherein the arm is radially resilient to allow the plunger engagement portion to become radially shifted from an engagement position where the retaining engagement is maintained, and into a release position where the retaining engagement is released.

In some embodiments the user operable trigger element is so configured that, when assuming the pre-firing state, the user operable trigger element engages the arm of the retaining element to prevent the arm from moving away from the engagement position. The user operable trigger element may be configured so that in the pre-firing state, e.g. in a pre-firing position, the user operable trigger element cooperates with the arm to prevent the arm from moving radially outwards away from a radially inwards holding position in which the arm engages the retaining geometry of the plunger. In such embodiments, upon triggering of the autoinjection device, the user operable trigger element is moved into the firing state, i.e. a firing position, wherein the user operable trigger element enables the arm to move radially outwards away from the retaining geometry of the plunger.

In some embodiments, the user operable trigger element is arranged to move axially, such as movable in the proximal direction, from a pre-firing position to a firing position, i.e. when shifted from the pre-firing state to the firing state. In other embodiments, the user operable trigger element is arranged to move rotationally from a pre-firing position to a firing position when shifted from the pre-firing state to the firing state.

In some embodiments, the power base and the housing comprises cooperating snap geometries for releasably retaining the power base in the first pre-firing position, said cooperating snap geometries being configured to release due to the force of the drive spring upon release of the retaining engagement.

In some embodiments the autoinjection device further comprises a biasing means providing a resilient biasing force on the power base urging the power base distally and away from the second fired position when the trigger element assumes the pre-firing state.

The drive spring may in some embodiments be configured to drive the piston from a piston start position to a piston end position, and wherein the drive spring provides a force Fₘᵢₙ onto the plunger when the piston assumes the end position, and wherein the resilient biasing force provided onto the power base is smaller than the force Fₘᵢₙ.

In some embodiments the sensor of the electronic module comprises a switch that senses the shift of position of the power base as it moves from the first pre-firing position into the second fired position.

In particular embodiments the switch comprises a dome switch having a dome positioned for engagement by the power base. The said resilient biasing force may be provided by the dome switch.

In some forms the power base defines a switch actuator configured to actuate the switch of the electronic module upon the power base being moved into the second fired position.

In other embodiments, the sensor of the electronic module comprises one or more of the sensors selected from the group consisting of an optical sensor, a force sensor, a magnetic sensor, an inductive sensor and an electrical conductive sensor.

In some embodiments, the electronic module is provided as a self-contained electronics assembly, the electronic module being coupled to or received within the proximal end of the housing.

In some variants, the electronic module comprises an energy source and a processor coupled to the energy source and the sensor, the processor being configured to register triggering of the injection device by means of the sensor sensing the shift of position of the power base as it moves from the first pre-firing position into the second fired position.

In some forms, the electronic module comprises timing means, and wherein the processor is configured to operate the timing means, such as a timer, to monitor the duration that the power base assumes in the second fired position, and wherein the registering triggering of the autoinjection device is made only if said duration is longer than a pre-defined time limit. In some forms, the predefined time limit is longer than 0.5 secs, such as longer than 1 sec, such as longer than 2 secs.

In some embodiments, the processor is configured to register a timing parameter associated with the triggering of the injection device, such as the time elapsed since registering triggering.

In some variants, the electronic module comprises a wireless communication interface configured to communicate with an external electronic device.

In some embodiments the autoinjection device comprises a needle shroud being axially movable relative to the housing, and a needle shroud spring which is arranged biasing the needle shroud in the distal direction. The needle shroud is movable from a first distal extended position into a proximal collapsed position when a proximally directed force is applied to the needle shroud, and from the proximal collapsed position into a distal extended locked position. The trigger element may be provided so that it couples to the needle shroud so that the trigger element changes from the pre-firing state to the fired state in response to the needle shroud being moved from the first distal extended position into the proximal collapsed position.

In further embodiments, when the needle shroud moves from the first distal extended position towards the proximal collapsed position the needle shroud causes the trigger element to shift from the pre-firing state into the fired state in a manner so that the needle shroud slaves the trigger element from a pre-firing position into a fired position.

In a second aspect the present invention relates to an autoinjection device for expelling a dose of drug from a held drug container, the injection device comprising:
a housing having a proximal end (P) and a distal end (D),
a drug container comprising a container barrel and a piston that is sealingly and slideably arranged inside the container barrel,
an injection needle connected to or connectable to a distal end of the drug container,
a plunger adapted for cooperation with the piston to drive the piston distally along a central axis, the plunger comprising a retaining geometry,
an energy source coupled to the plunger and providing a force on the plunger in a distal direction,
a plunger retaining arrangement comprising a retaining element that engages with the retaining geometry to retain the plunger in a pre-firing position, the retaining element being movable in a radial direction to release said engagement,
a user operable trigger element cooperating with the retaining element and shiftable from a pre-firing condition wherein the trigger element cooperates with the retaining element to maintain retaining engagement with the retaining geometry of the plunger, and into a firing condition wherein release of the retaining engagement is initialised,

wherein the autoinjection device defines a memory element being movable from a pre-firing position to a fired position, the memory element comprising an engagement surface configured for sliding engagement with an activation surface of the retaining element, wherein at least one of the engagement surface and the activation surface includes a surface being inclined relative to said radial direction, and

wherein, upon the trigger element being shifted from the pre-firing condition to the firing condition, the energy source acts on the plunger to force the retaining element radially to release the retaining engagement, the radial movement of the retaining element in turn forcing the memory element to move into the fired position by sliding engagement between the activation surface of the retaining element and the engagement surface of the memory element, said movement of the memory element being induced by said surface being inclined relative to said radial direction.

As the energy source acts to force the memory element to move into the fired position the autoinjection device becomes less dependent on tolerance variations so that the correct functions of a secondary function controlled by the position of the memory element becomes more reliable. In addition, the movement of the memory element into the fired position means that the movement of the memory element becomes less dependent on how the user operates the device.

In some embodiments, the memory element, when assuming the fired position, controls a secondary function of the autoinjection device, wherein said secondary function is distinct from the function associated with the release of the plunger, i.e. the release being provided by the retaining element being moved in the radial direction to release the engagement with the retaining geometry of the plunger. A non-exhaustive list of secondary functions may include one or more of controlling initialisation or generation of a feedback signal, i.e. conditional to the release of the plunger, such as a visible, audible or tactile signal, or generation of an electronic signal to be recorded or stored in electronic circuitry, wherein the electronic signal is responsive to the release of the plunger. Still other secondary functions may include a latch function, such as for locking a needle shroud in a particular position, i.e.

conditional to the release of the plunger.

In some embodiments the memory element is axially movable, and wherein said at least one surface is inclined relative to said radial direction and so oriented as to induce axial movement of the memory element from the firing position to the fired position upon radial movement of the retaining element.

In other embodiments the memory element is rotationally movable, and wherein said at least one surface is inclined relative to said radial direction and so oriented as to induce rotation of the memory element from the firing position to the fired position upon radial movement of the retaining element.

In some embodiments the retaining element includes a retaining surface that engages a cooperating surface of the retaining geometry to retain the plunger in the pre-firing position, and wherein one or both of the retaining surface and the cooperating surface include(s) a surface being inclined relative to said radial direction so that distal movement of the plunger, upon initial release of the retaining engagement, induces radial movement of the retaining element to disengage the retaining surface from the cooperating surface of the retaining geometry .

In some embodiments the trigger element assumes a pre-firing position when the trigger element assumes the pre-firing condition, and assumes a firing position when the trigger element assumes the firing condition, and wherein the trigger element cooperates with the retaining element to initiate release of the retaining engagement when the trigger element assumes the firing position.

In some embodiments the trigger element defines said memory element, and wherein the trigger element is movable from the pre-firing position to the firing position, and further to the fired position.

In some embodiments, the trigger element is movable axially from the pre-firing position to the fired position, such as movable proximally from the pre-firing position to the fired position. In some embodiments, the firing position is positioned at an intermediary position between the pre-firing position and the fired position.

In other embodiments, the trigger element is rotationally movable from the pre-firing position to the firing position. In some embodiments wherein the trigger element defines said memory element the trigger element is rotationally movable from the pre-firing position to the firing position, and further rotationally movable into the fired position. In these embodiments, the at one or both of the engagement surface and the activation surface include(s) a surface being inclined relative to said radial direction and being so oriented that radial movement of the retaining element induces rotation of the trigger element. The sequence of the rotation is initiated by the user which initially drives the trigger element to rotate from the pre-firing position to the firing position. Thereafter, the trigger element is in the first place urged to move by distal movement of the plunger, as forced by the energy source, which in turn induces radial movement of the retaining element, and which in turn induces rotation of the trigger element from the firing position to the fired position.

Some embodiments of the autoinjection device comprises a needle shroud being axially movable relative to the housing, and a needle shroud spring which is arranged biasing the needle shroud in the distal direction, wherein the needle shroud is movable from a first distal extended position into a proximal collapsed position when a proximally directed force is applied to the needle shroud, and from the proximal collapsed position into a distal extended locked position, and wherein the trigger element couples to the needle shroud so that the trigger element moves from the pre-firing position to the fired position in response to the needle shroud being moved from the first distal extended position into the proximal collapsed position.

In some embodiments, the first distal extended position is the same as the distal extended locked position. In other embodiments, the first distal extended position may be located distally or proximally relative to the distal extended locked position. The distal extended locked position defines a state wherein the needle shroud protects the needle from being touched by the user. The proximal collapsed position defines a state wherein the needle extends distally beyond the needle shroud, or where the needle is positionable to extend distally beyond the needle shroud, to allow for insertion of the needle into an injection site.

The needle shroud may be configured so that when it moves from the first distal extended position towards the proximal collapsed position the needle shroud causes the trigger element to move from the pre-firing position into the fired position, the needle shroud slaving the trigger element into the firing position, and optionally into the fired position.

In some embodiments a latch is associated with the trigger element, the latch engaging when the trigger element assumes the fired position to arrest the trigger element in the fired position.

The latch may in some embodiments be provided by cooperating latch geometries of the trigger element and the housing to prevent the trigger element from being moved distally away from the fired position. In other embodiments the latch is configured to rely on a frictional coupling, such as a frictional engagement, between the trigger element and the housing to prevent the trigger element from being moved distally away from the fired position. In some embodiments the latch is configured to provide a permanent axial locking of the trigger element relative to the housing when the trigger element assumes the fired position.

In some embodiments, when the needle shroud moves from the proximal collapsed position into the distal extended locked position, the needle shroud moves relative to the arrested trigger element, and wherein the needle shroud cooperates with the trigger element to lock the needle shroud as the needle shroud is moved distally into the distal extended locked position.

In some embodiments at least one of the needle shroud and the trigger element comprises a lock element which is resiliently urged towards the other of the needle shroud and the trigger element to move along relative to a surface of said other of the needle shroud and the trigger element when the needle shroud moves relative to the arrested trigger element until the lock element reaches a locking geometry formed in or on said other of the needle shroud and the trigger element upon the needle shroud being moved distally into the distal extended locked position so as to lock the needle shroud in the distal extended locked position.

In some embodiments the trigger element includes a distally directed lock surface configured to engage a proximally directed locking geometry of the needle shroud to prevent the needle shroud to be moved proximally when the needle shroud assumes the distal extended locked position.

In some embodiments the distally directed lock surface is formed on a resiliently movable lock element being movable from a non-locking position into a locking position, and wherein a biasing means urge the resiliently movable lock element towards moving to the locking position, and wherein the resiliently movable lock element is moved from the non-locking position into the locking position upon the needle shroud being moved from the proximal collapsed position into the distal extended locked position.

In some embodiments the resiliently movable lock element is configured to slide along a sliding surface of the needle shroud as the needle shroud is moved from the proximal collapsed position into the distal extended locked position for the distally directed lock surface of the trigger element to axially align with the proximally directed locking geometry of the needle shroud to enable the distally directed lock surface to engage with the proximally directed locking geometry.

In some embodiments the resiliently movable lock element is structured as a lock sleeve.

Some embodiments of the autoinjector forms a device wherein the energy source comprises a helical compression spring arranged in a pre-tensed state exerting a distally directed force on the plunger.

In further embodiments a latch is associated with the memory element, the latch engaging when the memory element assumes the fired position to arrest the memory element in the fired position.

The latch may in some embodiments be provided by cooperating latch geometries of the memory element and the housing to prevent the memory element from being moved distally away from the fired position. In other embodiments the latch is configured to rely on a frictional coupling, such as a frictional engagement, between the memory element and the housing to prevent the memory element from being moved distally away from the fired position. In some embodiments the latch is configured to provide a permanent axial locking of the memory element relative to the housing when the memory element assumes the fired position.

In some embodiments of the autoinjector, the device irreplaceably accommodates a container within the housing so that the container cannot be removed from the device without the use of tools. In such embodiments, the autoinjector forms a disposable device.

In some embodiments the container is provided as a syringe having a barrel and with an injection needle fixedly attached to the barrel.

In embodiments incorporating a cartridge and a separate needle unit, the cartridge and the needle unit may be initially held in a configuration where the cartridge and the needle unit are separated by a distance. The energy source may be capable, upon release of the plunger retaining arrangement, to cause the cartridge and the rear needle to enter into the state where the cartridge septum is pierced by the rear needle and subsequently to cause the plunger to move to dispense the drug through the needle.

In some embodiments the needle or needle unit substantially follows movement of the housing as the housing moves relative to the needle shroud. In particular embodiments, the needle or needle unit is attached to the housing in a way preventing relative axial movement between the housing and the needle.

Any of the features and embodiments listed in relation to the second aspect of the invention may in further embodiments combine with the features and embodiments listed in relation to the first aspect of the invention.

In a third aspect the present invention relates to an autoinjection device for expelling a dose of drug, the injection device comprising:
a housing having a proximal end (P) and a distal end (D),
a drug container comprising a container barrel extending along an axis, a distal outlet connectable or connected to an injection needle, and a piston that is sealingly and slideably arranged inside the container barrel,
a power unit configured for driving the piston distally along the axis to expel a drug contained in the drug container, the power unit comprising:
   a plunger adapted for cooperation with the piston to drive the piston distally along a central axis, the plunger comprising a retaining geometriy and defining one or more radially protruding click protrusions,
   a drive spring arranged in a tensed state wherein a first end portion of the drive spring acts on the plunger with a force biasing the plunger distally, and
   a power base operably coupled to the drive spring and the plunger, the power base grounding a second end portion of the drive spring, wherein, in a pre-firing state, a retaining element of the power base releasably engages the retaining geometry of the plunger to retain the plunger against the force of the drive spring, the power base further comprising a resilient click arm configured to cooperate with said one or more click protrusions of the plunger, the click arm being moved radially to provide a click noise for each click protrusion that passes the click arm during the expelling movement,

and
a user operable trigger element cooperating with the retaining element and shiftable from a pre-firing state wherein the trigger element cooperates with the retaining element to maintain retaining engagement with the retaining geometry of the plunger, and into a firing state wherein said retaining engagement is released,

wherein the injection device further comprises a sensor system including a sensor and a processor operably coupled with the sensor, the sensor system configured to detect radial movement of the click arm, wherein the sensor system is configured for generating a signal for each click protrusion that passes the click arm during expelling movement and configured for recording each said signal.

In such device, real time monitoring of operation of the device is provided facilitating improved control and user interaction with the device during drug administration. Detection of an End-of-Dose state can be more reliably detected as the number of clicks provides a means for determining dose expelling rate. In combination with the recording of a predetermined number of click sounds required for expelling the full dose this expelling rate may be utilized for determining the correct End-of-Dose state. Furthermore, by registering a series of dose clicks, which will generate signals in a pre-defined sequence, it becomes possible to distinguish true dose expelling signals from potential unwanted signals, such as signals generated by unintentionally dropping the device on a hard surface.

In some embodiments the plunger comprises a spring seat, and wherein the drive spring is a compression spring having the second end of the drive spring grounded by the power base and the first end providing a distally directed force on the spring seat of the plunger

In some embodiments the plunger is hollow, wherein the drive spring is a compression spring, and wherein the compression spring extends at least partly into the hollow plunger. In other embodiments, the drive spring is provided as a helical compression spring arranged to encircle at least a portion of the plunger. When the drive spring of the power unit is arranged in the tensed state, the compression spring is arranged in a compressed state and thus urges the ends of the drive spring away from each other.

In some embodiments, the sensor is configured as a deflectable transducer that deflects as the click arm deflects radially. The transducer may comprise a strain sensitive material. The strain sensitive material may be adhered to or otherwise disposed on the click arm. In other embodiments, the strain sensitive material is provided on a carrier foil wherein the carrier foil includes a portion that is mechanically adhered to or retained relative to at least a portion of the click arm.

In some embodiments, the deflectable transducer defines a base portion attached to a base portion of the click arm, and further defines a deflectable portion having a tip end, wherein the deflectable portion deflects relative to the base portion upon the click arm cooperating with the click protrusions.

The deflectable transducer may be provided to comprise: a) a carrier foil that extends from the base portion to the tip end of the deflectable portion, and b) a sensor element comprising the strain sensitive material disposed on the carrier foil and extending from the base portion towards the tip end of the deflectable portion, wherein the carrier foil, between the base portion and tip end of the deflectable portion, comprises a non-supported portion which is not contacting the click arm, and wherein said strain sensitive material disposed at least partly along the non-supported portion. The tip end may in some embodiments be provided so that it is not fixedly attached by the deflectable end of the click arm, but only supported by the click arm as the click arm moves from a radially inwards position to a radially outwards strained position.

By forming the carrier foil to be non-supported by other components along a portion of its extension, i.e. except for the strain sensitive material itself, the strain sensitive material disposed along the non-supported portion of the carrier foil is exclusively supported by the carrier foil. Due to the non-supported portion of the carrier foil, a high degree of strain at locations where the strain sensitive material is disposed can be provided. As a result, superior signal amplitudes can be obtained resulting in high signal to noise ratios to be obtained. Also, the improved signal amplitudes can be utilized for particular purposes such as waking up a dormant or sleeping micro-processor. Bending out the substrate itself and releasing it quickly gives a high voltage output, which is easily distinguished from noise in the system. The substrate forms a very thin and short beam and, therefore, it becomes less sensitive to vibrations in the injection device which may occur if the device is being unintentionally dropped on a hard surface.

In further embodiments, the said click arm provides a first click arm and wherein the power base comprises one or more additional click arms configured similarly to the first click arm to cooperate with click protrusions of the plunger, and wherein the plurality of click arms are arranged symmetrically around the plunger. The number of click arms may in different embodiments be one, two, three, four or more individual click arms. The click arms may in certain embodiments be provided as axially extending arms which generally runs parallel with the axis. In certain embodiments, each of the click arms extends distally from a proximally arranged transverse section. In other embodiments, each of the click arms extends proximally from a distally arranged tubular section of the power base. In some embodiments respective ones of the one or more retaining elements, such as being provided as retaining arms, is defined by one of said click arms. In other embodiments the one or more click arms are formed individually from the one or more retaining elements.

In some embodiments, the device comprises a plurality of sensors each cooperating with a respective one of the click arms. In embodiments wherein at least two click arms are disposed symmetrically around the axis to provide substantially simultaneous clicks for each click protrusion that passes the click arms during the expelling movement, the sensor system may be configured to compare individual signals from each sensor, and omit recording of sensor signals when the individual signals differ from each other, such as when differing from each other above a predetermined signal level, and/or differing from each other beyond a predefined threshold time interval.

Any of the features and embodiments listed in relation to the third aspect of the invention may in further embodiments combine with the features and embodiments listed in relation to the first and second aspects of the invention.

As used herein, the term “drug” is meant to encompass any drug-containing flowable medicine or combinations of separately held plurality of drug-containing flowable medicines capable of being passed through a delivery means such as a cannula or hollow needle in a controlled manner, such as a liquid, solution, gel or fine suspension.

### DETAILED DESCRIPTION OF THE INVENTION

The invention will now be described in further detail with reference to the drawings in which:

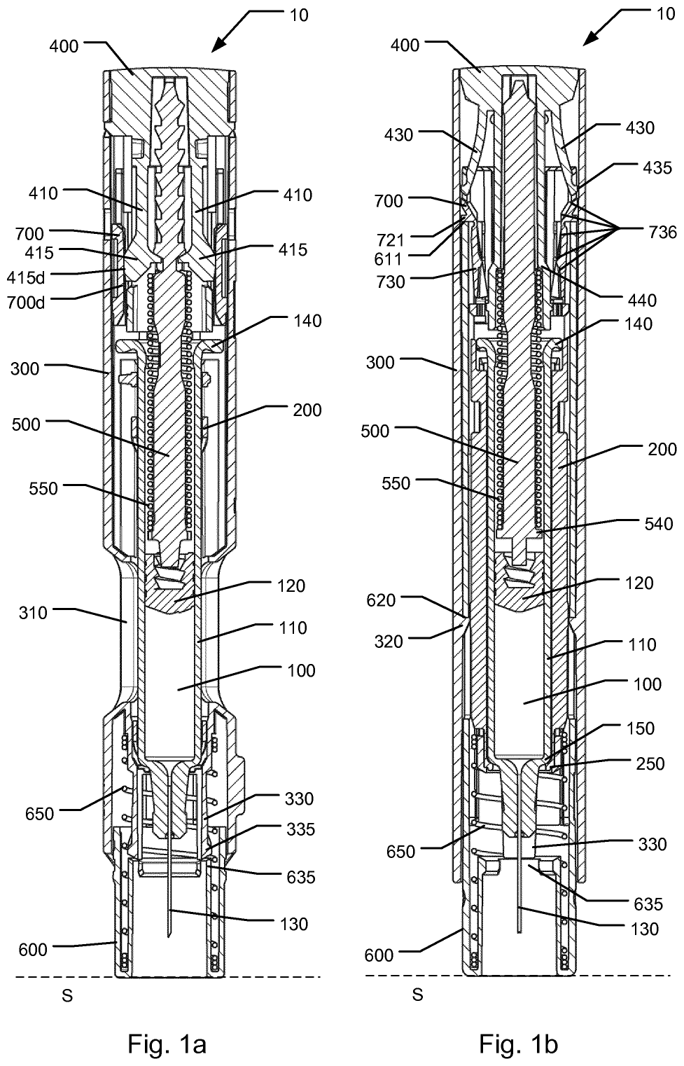

FIGS. 1a and 1b show sectional front and side views of a state of the art autoinjection device 10, the device being in a state where a needle shroud is fully extended and protects the needle of a held syringe,

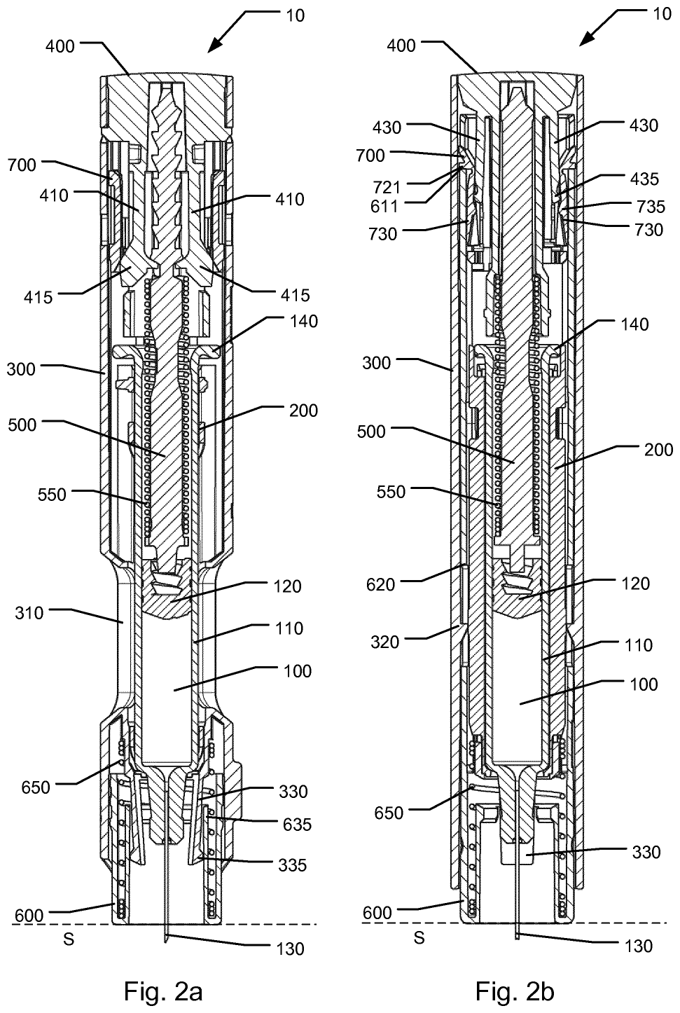

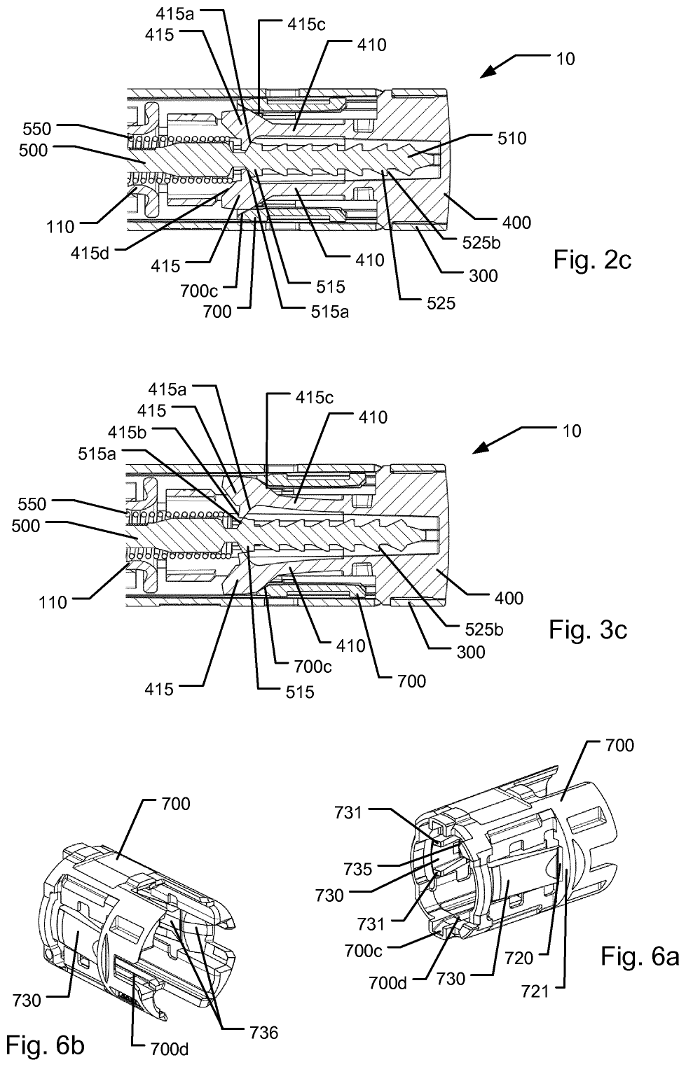

FIGS. 2a and 2b show sectional front and side views of the device 10 illustrating a state where the device has been pressed onto an injection site S and where an injection needle of a syringe initially protrudes from the needle shroud,

FIG. 2c is a detailed magnified view of FIG. 2a, showing the proximal portion of the device 10,

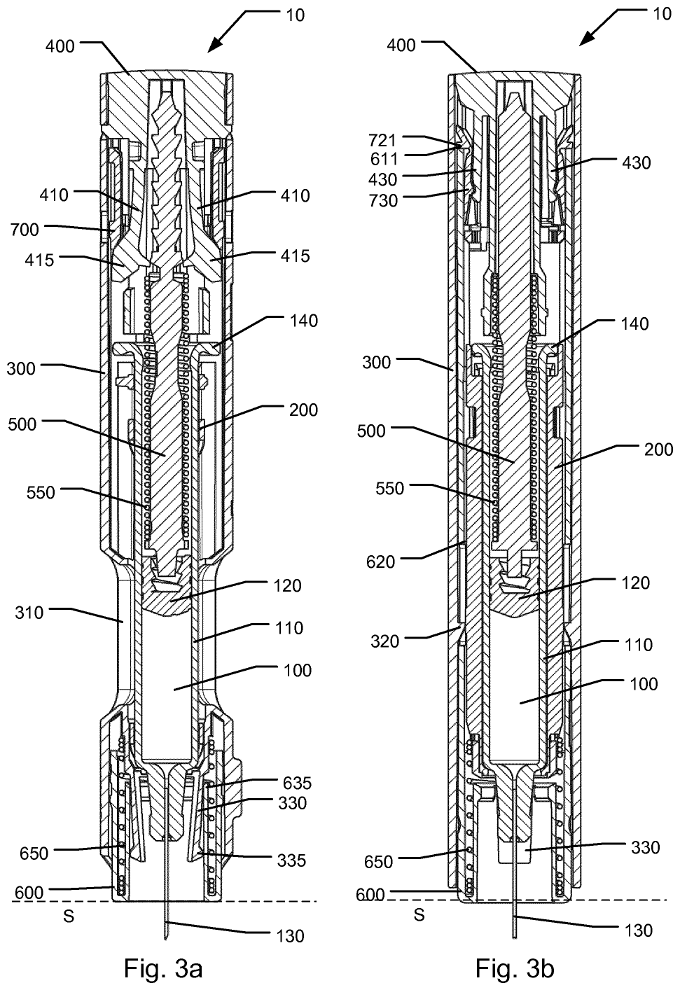

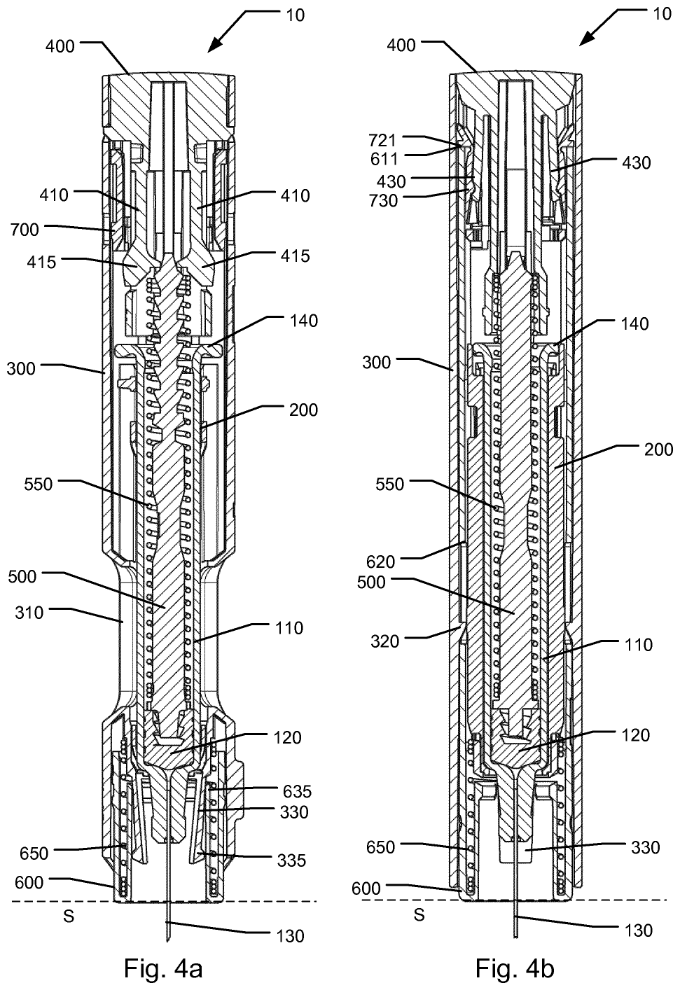

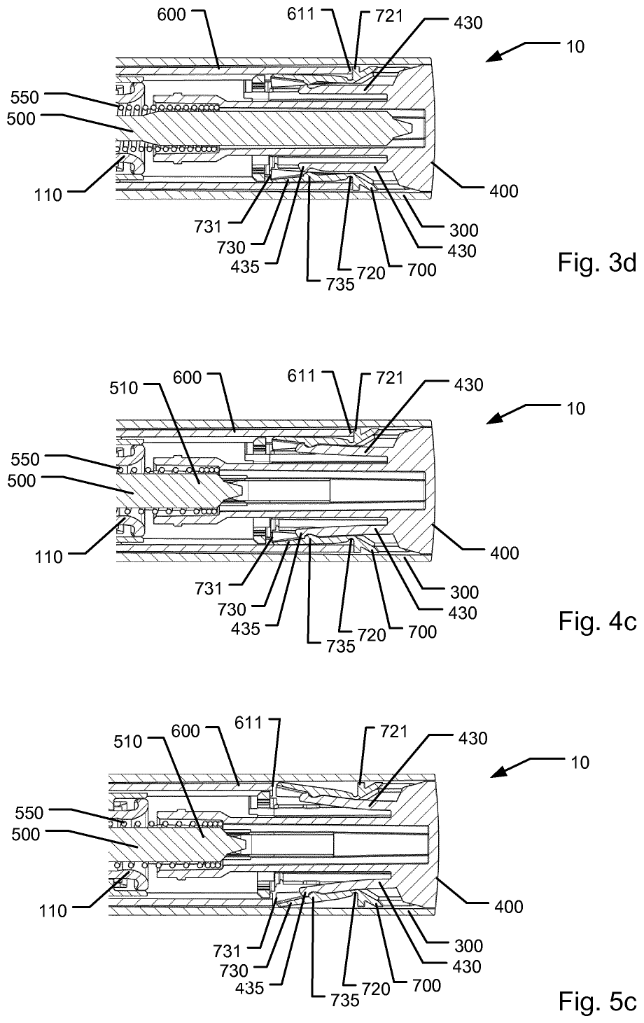

FIGS. 3a and 3b show sectional front and side views of the device 10 illustrating a state slightly before the needle protrudes fully from the needle shroud and wherein release of a plunger is about to be initiated,

FIG. 3c is a magnified view of FIG. 3a, showing the proximal portion of the device 10,

FIG. 3d is a detailed magnified view of the proximal portion of the device 10 in a view generally corresponding to the view shown in FIG. 3b but in a state just prior to the state shown in FIGS. 3a and 3b,

FIGS. 4a and 4b show sectional front and side views of the device 10 illustrating a state after the plunger has been released and where the drug of a held syringe has been expelled,

FIG. 4c is a detailed magnified view of FIG. 4b, showing the proximal portion of the device 10,

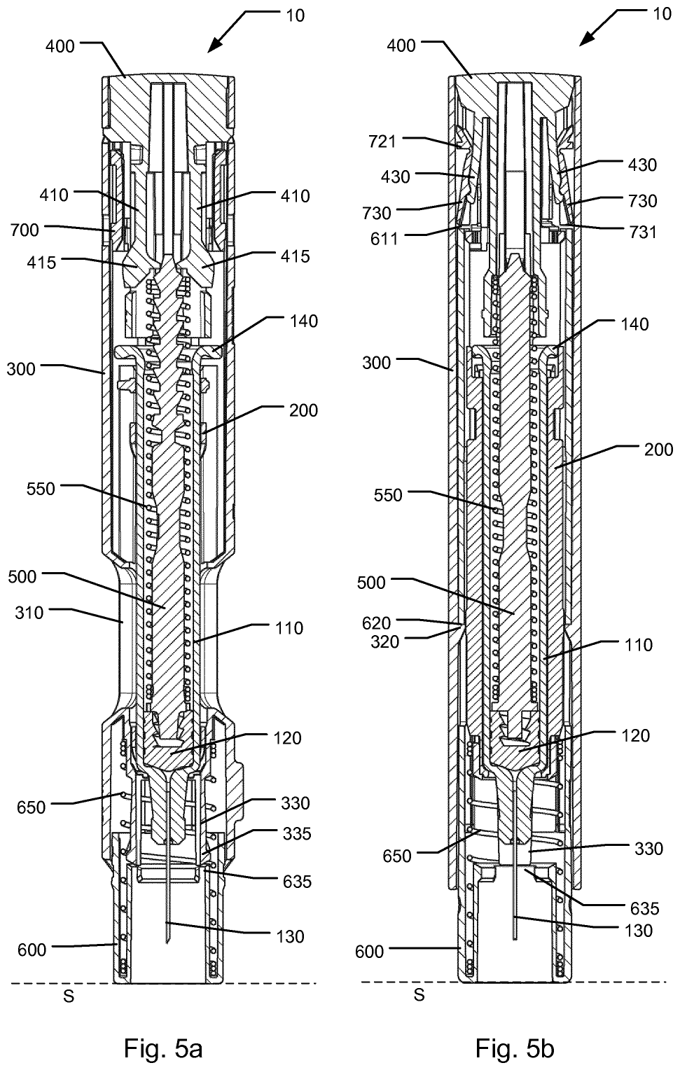

FIGS. 5a and 5b show sectional front and side views of the device 10 illustrating a state where the device has been lifted relative to the injection site S and wherein the needle shroud assumes a locked extended state,

FIG. 5c is a detailed magnified view of FIG. 5b, showing the proximal portion of the device 10,

FIGS. 6a and 6b show perspective proximal and distal views of trigger element 700 of the injection device 10,

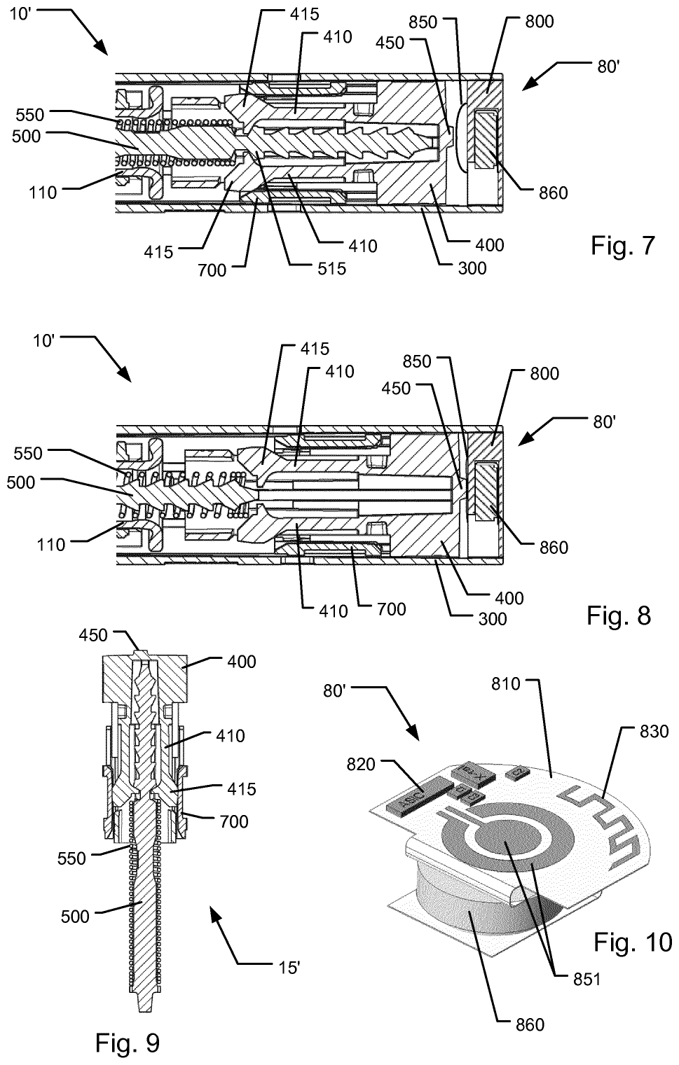

FIG. 7 is a sectional side view of the proximal portion of a first exemplary embodiment of an autoinjection device 10′ according to the invention, the device being in a state prior to triggering,

FIG. 8 shows a view corresponding to the view in FIG. 7, but wherein the autoinjection device 10′ is in a state after triggering, i.e. wherein the plunger has been released and where the drug of a held syringe has been expelled,

FIG. 9 shows a sectional side view of a power unit 15′ of the autoinjection device 10′ of FIG. 7 before insertion into housing 300,

FIG. 10 shows schematically a perspective view of main components of an electronic module 80′ before coupling to housing 300,

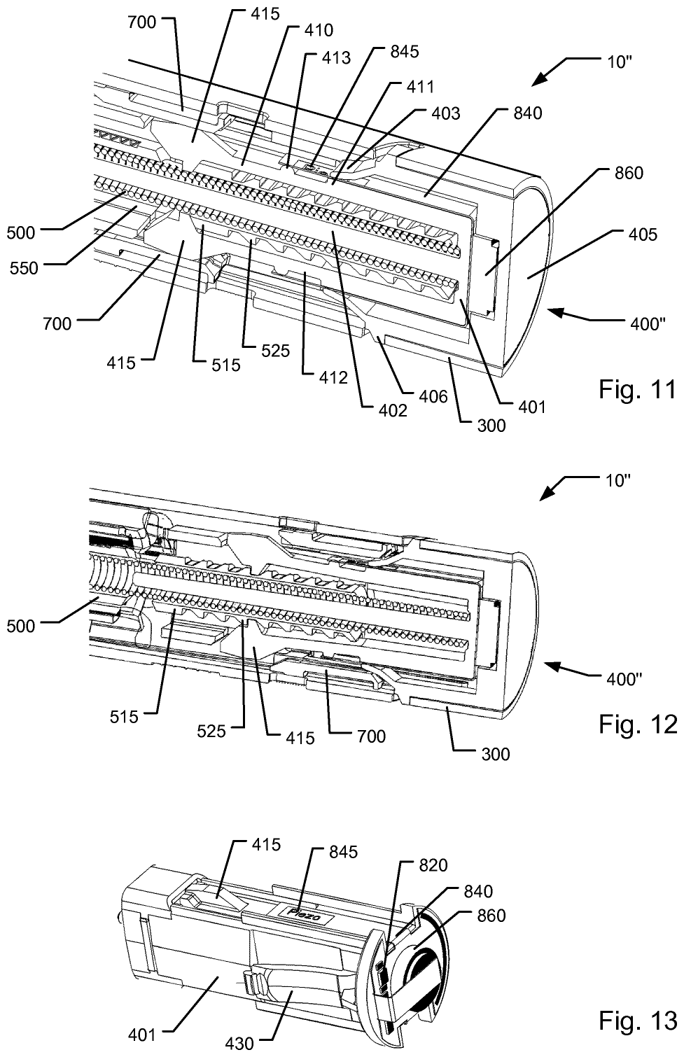

FIG. 11 is a cross-sectional perspective view of a second exemplary embodiment of an autoinjection device 10′ having a power base assembly 400″ including electronic dose sensing circuitry,

FIG. 12 is a cross-sectional perspective view of the device 10″ shown in FIG. 11, wherein the view depicts a state during expelling, the device being in a state prior to triggering, and

FIG. 13 shows a perspective view of selected parts of power base assembly 400″ of the device shown in FIG. 11 before coupling to housing 300.

### DESCRIPTION

In the context of the present disclosure it may be convenient to define that the term “distal end” in the appended figures is meant to refer to the end of the injection device which carries the injection needle whereas the term “proximal end” is meant to refer to the opposite end of the injection device pointing away from the injection needle. The shown figures are schematical representations for which reason the configuration of the different structures as well as the relative dimensions are intended to serve illustrative purposes only.

With reference to FIGS. 1a through 6b the following is a description of a state of the art medical autoinjection device 10 for administering a pre-determined amount of a liquid medicament. As will be described later, the general operating principle of the shown device 10 may be modified enabling registering of triggering of the injection device by means of an electronic module. However, the electronic module is not shown in the embodiment shown in figs. la through 6b but will be more thoroughly described in connection with FIGS. 7 through 10.

The shown device 10 is a disposable autoinjector configured for expelling a dose of a drug in a single administration whereafter the device 10 is ready for disposal. FIGS. 1a through 5c show various states of the injection device 10 during operation thereof with different views offering a detailed assessment of the operating principle.

Referring to FIGS. 1a and 1b, injection device 10 includes an elongated housing 300 that extends along a central longitudinal axis, housing being configured for being gripped by the palm of the user. The housing 300 forms a tubular shell which is closed off at the proximal end by a cap which in the following will be referred to as a power base 400. During assembly the power base 400 snaps into the housing 300 by means of snap protrusions which are received in recesses or openings to provide a fixed non-releasable mounting of power base 400 within the proximal end of the housing 300.

At the distal end of the housing 300 a protective cap (not shown) will normally be arranged to cover a needle arrangement located at the distal end of the housing.

In the shown embodiment 10, the housing 300 accommodates a standard prefilled syringe (PFS) as widely used in industry. The syringe 100 comprises a tubular barrel 110 having a neck portion 115 located distally wherein the neck portion 115 has a reduced diameter compared to the diameter of the barrel 100. An injection needle 130 is mounted to the neck portion 115 and a removable cap (not shown) provided in the form of a rigid needle shield (RNS) will prior to use be attached to the neck 115 so that the needle shield sealingly and sterilely seals off the needle 130 Internally in the barrel 110 a slideably arranged piston 120 is arranged. A drug may be accommodated within the barrel between the piston 120 and the needle 130. Although the shown syringe only incorporates a single piston 120, other configurations may incorporate multiple pistons for accommodation and expelling of one or more drugs, including drugs to be reconstituted before administration. In other not shown embodiments, instead of a PFS type syringe, the housing may alternatively include other types of medicament containers, such as a cartridges configured to receive a separate injection needle.

Injection device 10 will typically be available in a form which further includes a removable protective cap (not shown) that attaches to a distal end of the device 10 to protect a needle end of the device 10. As commonly known for autoinjectors that incorporate a PFS syringe having an RNS shield attached, the protective cap may couple to the RNS so that the RNS is removed together with the protective cap. This situation is depicted in the state shown in FIGS. 1a and 1b.

In the shown embodiment, a syringe holder 200 is arranged to hold syringe 100 inside housing 300 in a manner so that syringe 100 is fixedly withheld within the housing 300 by means of the syringe holder 200. Syringe holder 200 includes a body extending along a central longitudinal axis and being adapted to receive the barrel 110 of syringe 100. The body of the syringe holder 200 includes two longitudinal body sections disposed around the central longitudinal axis, where each of the body sections has a distal end with a radial inwards flange section 250 adapted for being received in a circumferential gap between the shoulder section 150 of barrel and the not shown RNS covering the needle. In this way the syringe holder 200 retains the syringe 100 so as to prevent the syringe from moving distally relative to the syringe holder 200. The two longitudinal body sections of syringe holder 200 are connected to each other by means of flexible portions allowing the two body sections to be radially moved away from each other in order to insert the syringe with the RNS attached into syringe holder 200. During manufacture, the assembly formed by the syringe holder and the syringe with the RNS attached is insertable into housing 300 through a proximal opening in the housing shell.

The lower distal half of the housing 300 includes two opposing window openings 310 allowing visual inspection of the drug contained within the syringe of the device 10. In addition, window openings 310 allow a user of the device to determine whether or not the device 10 has been used for an injection by inspecting the presence or the location of a piston of syringe 100. In the course of an injection, window openings 310 also allow for a rod-shaped plunger 500 of the device to become increasingly visible by the plunger gradually blocking more and more of the space between window openings 310.

FIGS. 1a and 1b show front and side sectional views of the device 10 after the protective cap has been removed but in a condition prior to the administration operation. Shown protruding from the distal end of housing 300 is a needle shroud 600 which is received partly within and arranged coaxially and axially slidable relative to housing 300. a needle shroud spring 650 is arranged biasing the needle shroud 600 in the distal direction. Needle shroud 600 is movable, when a proximally directed force is applied to the needle shroud 600, from a first distal extended position (shown in FIGS. 1a and 1b) and into a proximal collapsed position (shown in FIGS. 4a and 4b). Upon release of the proximally directed force, the needle shroud spring 650 pushes needle shroud 600 from the proximal collapsed position into a distal extended locked position (shown in FIGS. 5a and 5b).

The injection device 10 is configured for being triggered to expel a dose when the needle shroud 600 is moved from the distal extended position towards the proximal collapsed position. As the syringe 100 is substantially fixedly mounted within housing 300 of the device 10, the injection needle 130 follows axial movement of the housing when the housing is moved relative to the needle shroud 600.

The protective cap, when attached to injection device 10, prevents the needle shroud 600 from being manipulated and thereby prevents premature unintentional triggering of the injection device 10. In the shown embodiment, this function may be provided by a mechanism incorporating radially flexible arms 330 formed in the housing, the flexible arms having heads 335 formed at an internal location in the housing 300 arranged to cooperate with an internal skirt 635 provided inside the needle shroud 600. The heads 335 and skirt 635 are located at the same radial position. Thus for the needle shroud 600 to become pushed proximally relative to housing 300, the flexible arms 330 with heads 335 are required to become deflected radially inwards by cooperating with skirt 635 before the skirt and thus the entire needle shroud is movable away from the distal extended position. As long as the RNS and/or the protective cap is still attached to the syringe the flexible arms 330 with heads 335 are initially blocked against moving radially inwards by the presence of the RNS. The skirt 635 is thus not able to axially pass the heads 335. Only after removal of the protective cap with the RNS and forcing the needle shroud 600 towards the proximal collapsed position the heads will cooperate with skirt 635 to move the heads radially inwards and allow the skirt 635 to pass the heads of the flexible arms (cf. FIG. 2a).

Piston 120 is driveable towards the needle outlet in order to dispense medicament from the syringe 100. The dispensing is carried out by an expelling assembly incorporating the plunger 500 and a pre-stressed drive spring 550.

In the shown embodiment, the needle shroud 600 forms a distal portion and a proximal portion. The distal portion is provided as a generally hollow tubular member having a distal end rim arranged to form an abutment surface, the tubular member initially covering the injection needle 130. The proximal portion of the needle shroud 600 forms two opposed axial running legs extending from the distal portion and in the proximal direction for a substantive part of the length of the housing. Each of the two opposed axial running legs ends in a proximally facing abutment surface 611. The needle shroud 600 with its two legs is shaped to be accommodated within the housing 300 with the radial outer surface of the needle shroud being in intimate but slideable contact with a radially inwards facing cylindrical surface of the housing shell.

The needle shroud 600 cooperates with a trigger element 700 which is located at the proximal end of the needle shroud 600. Trigger element 700 serves as a memory element which assumes a first distal position prior to use of the device 10, and which assumes a second proximal pre-defined parked position after the device 10 has been fully triggered, and wherein the memory element stays in the parked position subsequent to triggering. In the shown embodiment the trigger element both serves as a trigger sleeve, and also serves as a lock sleeve for the needle shroud. For accommodating both functions, the trigger element 700 is movable axially in the proximal direction relative to the housing 300 from a pre-firing position (FIGS. 1a and 1b) to a firing position (FIGS. 2a and 2b), and further to a fired position (FIGS. 4a and 4b). Trigger element 700 is formed as a generally tubular hollow member. Initially, before triggering, and until the needle shroud 600 is pushed distally after expelling, a proximal portion of the legs of the needle shroud 600 is arranged in axially overlapping relationship with the trigger element 700. In this relative position the trigger element 700 is accommodated radially between the two opposed axial running legs of the needle shroud 600 so that an inner surface of each leg is in slideable contact with an internal surface of the trigger element 700. Referring to the state shown in FIG. 1b, and also referring to FIG. 6a, each proximally facing abutment surface 611 of the legs of the needle shroud 600 abuts a distal abutment surface 721 of the trigger element. Thus, when the needle shroud

In the shown embodiment, both the needle shroud 600 and the trigger element 700 are mounted in a way that prevents rotational movement but allows axial movement relative to the housing 300. The needle shroud 600 is urged in the distal direction by means of the needle shroud spring 650 so that when no externally applied force is exerted on the needle shroud, the needle shroud assumes its distal extended position which is shown in FIGS. 1a and 1b. In this position a stop geometry 620 on needle shroud 600 engages a stop 320 in the housing preventing the needle shroud 600 from moving further in the distal direction.

When an externally applied force is exerted on the needle shroud 600 for moving the needle shroud in the proximal direction relative to the housing, such as when device 10 is pressed with the needle shroud against an injection site, the externally applied force acts counter to the force provided by the needle shroud spring 650 resulting in the needle shroud 600 and the trigger element 700 being forced to move in the proximal direction relative to the housing. When the needle shroud 600 assumes the proximal collapsed position a proximal facing surface of the trigger element 700 prevents the trigger element and thus the needle shroud 600 from moving further proximally relative to the housing.

As the device 10 is removed from the injection site, the needle shroud 600 will move distally due to the force from the needle shroud spring 650. After an injection has been performed, as the needle shroud 600 reaches its distal extended position again, as shown in FIGS. 5a and 5b, it will be locked in this position to render the needle shroud inoperable (to be further explained below). While referring to “its distal extended position” it is to be noted that the shown device 10 is so designed that the said distal extended position where the needle shroud is made inoperable corresponds to the initial distal extended position the needle shroud assumes prior to triggering. However, in other embodiments, the final distal extended position where the needle shroud is made inoperable may be located slightly different than the initial distal extended position prior to triggering, e.g. positioned at a position slightly proximally or slightly distally relative to the distal extended position shown in FIGS. 1a and 1b.

The needle 130 of syringe 100 is arranged at the distal end of the housing 300, such that the needle shroud 600 completely covers the needle when the needle shroud is in its distal extended position. When the needle shroud 600 is in its proximal collapsed position, the needle 130 protrudes through a central opening in the needle shroud 600.

The expelling assembly of injection device 10 is based on a plunger that is driven in the distal direction along the central longitudinal axis of the device for advancing the piston 120 to thereby expel the dose of drug accommodated within the syringe 100. The plunger 500 in the shown embodiment forms a solid rod having a circular flange arranged at the distal end of the plunger. In device 10 with the rod-shaped plunger 500 arranged along the central axis, a stored energy source in the form of a pre-stressed helical compression drive spring 550 is arranged to encircle the plunger rod 500 along a portion of its length. Drive spring 550 is energized by straining the compression spring during manufacture of the device. The distal end of drive spring 550 is supported onto plunger 500 by a circular flange arranged at the distal end of the plunger. The proximal end of drive spring 550 is supported by a spring seat (non-referenced) formed at a distal end of power base 400 and thus grounds the proximal end of drive spring relative to the housing 300.

As mentioned, in the shown embodiment, the drive spring 550 urges the plunger 500 in the distal direction. In the non-triggered state of the injection device 10, a plunger retaining arrangement associated with the housing engages with a retaining geometry of the plunger to retain the plunger 500 in a pre-firing position. In the shown embodiment, and referring to 1a, FIGS. 2c and 3c, the retaining arrangement comprises, on the plunger 500, a pair of stepped blocking geometries 515 proximally adjoining a recessed portion of the plunger 500. The plunger retaining arrangement further comprises two retaining elements in the form of two retaining arms 410 extending axially in the distal direction from the proximal portion of the power base 400. Each of the two retaining arms 410 forms a radially resilient arm that ends in an enlarged blocking head 415 having its radially inwards facing portion situated in the recessed portion of the plunger rod 500. Generally referring to FIG. 2c, with the plunger retaining arrangement assuming the state shown in FIG. 1a, an inclined proximal surface 415a of enlarged blocking head 415 engages a correspondingly inclined distal surface 515a, these surfaces thus forming retaining geometries of plunger 500. Hence, the force exerted by drive spring 550 acts to push the enlarged blocking heads 415 radially outwards. In the initial non-triggered state of the device 10, as shown in FIG. 1a, a radially outward facing surface provided on each of the enlarged blocking heads 415 engages a radially inwards surface 700d of the trigger element 700, the presence of the trigger element, when located in the pre-firing position, thus effectively prevents triggering of device 10.

Referring to FIG. 2c, each of the enlarged blocking heads 415 includes, at a radially outwards portion thereof, an inclined proximally facing surface 415c configured for sliding engagement with a corresponding inclined distal facing surface 700c provided at the distal end of the trigger element (see FIGS. 2c and 6a). During triggering, the trigger element 700 will initially be pushed proximally by the needle shroud 600, due to engagement between elements 611 and 721, and once the trigger element assumes the firing position shown in FIG. 2c, each of the proximally facing surfaces 415c of enlarged blocking heads 415 axial aligns and engages the respective inclined distal facing surfaces 700c of trigger element 700. Due to the inclination, the radially outwards force generated by the bias of the drive spring onto the plunger is transferred to the enlarged blocking heads 415 which in turn will act to urge the trigger element 700 further proximally meaning that the force provided by needle shroud 600 to initiate the trigger movement of trigger element 700 will be amplified by the force provided by the drive spring 550. This causes the trigger element to be effectively pushed proximally until the trigger element 700 assumes the fired position shown in FIG. 3c. During movement of trigger element 700 from the firing position shown in FIG. 2c and into the fired position shown in FIG. 3c, the axial movement of trigger element is likely to be accompanied by proximal movement of the needle shroud 600 due to the momentum of movement of the needle shroud during the triggering movement of needle shroud.

As shown in FIG. 3c, the state refers to a situation where, for each resilient arm 410, the inclined proximal surface 415a of enlarged blocking head 415 slips free from engagement relative to the inclined distal surface 515a on the plunger 500, and the plunger 500 is thus released to be driven forward by the drive spring 550.

Alternatively to using a pre-stressed spring which is compressed during manufacture of the device, other embodiments of autoinjectors may include a mechanism for compressing the spring as an initial procedure when putting the device into use. Also, the energy source may in other embodiments be provided as a torsion spring which is pre-stressed to exert a torsion force for driving forward a rotational drive of the expelling assembly. Alternatively, the energy source may be in the form of a compressed medium such as a gas. Still alternatively, the energy source may include a gas generator such as an electro-chemical cell.

Referring again to FIG. 2c, the plunger 500 furthermore provides, at its proximal portion, a series of teeth 525 each having a gradually rising slope and an abrupt decline 525b in the direction of relative movement. A radially inwards facing surface of the enlarged blocking head 415 of each resilient arm 410 is configured to sequentially cooperate by the inherent elastic properties of the resilient arm with each tooth 525 to generate an audible click as the enlarged blocking head passes each tooth. In the shown embodiment, each of the enlarged blocking heads 415 cooperate with six consecutive teeth. The enlarged blocking heads and the teeth thus generate progress clicks in the course of the dispensing procedure to signal expelling of liquid drug, and with the omission of a click to signify end of dosing. In the shown embodiment, two opposed retaining arms are provided in a symmetrical configuration, where the retaining arms cooperate with a corresponding number of protrusions or recesses formed on the plunger. In other embodiments a single arm may be provided necessitating a support surface of some kind arranged radially oppositely to the single arm. In still other embodiments, three or more arms may be provided, preferably being disposed symmetrically around the axis. In the shown embodiment, the plunger 500 is formed with surfaces configured for cooperation with engaging sliding surfaces of the power base 400, wherein the engaging surfaces are formed in a way that ensures that the plunger will not rotate relative to the power base at least as long as the plunger protrusions 525 generate click sounds during expelling.

In the following, the components that relate to the needle shroud lock function will be further described. Referring back to FIG. 1b, 5c and FIG. 6a/6b, the trigger element 700 includes two resiliently movable lock elements formed as a pair of deflectable lock arms 730 forming part of the needle shroud lock mechanism. When the needle shroud lock function is established, the deflectable lock arms 730 render the needle shroud 600 permanently arrested, i.e. when the needle shroud, subsequent to finalisation of an injection, is returned to the distal extended position. As shown in FIG. 6a, each of the deflectable arms 730 connects by means of a film hinge 720 to the remaining of the trigger element 700. Each of the deflectable arms 730 comprises a rigid beam section extending from the film hinge 720 to a free distal end comprising distally directed lock surfaces 731. The deflectable arms 730 are due to the film hinge 720 able to be moved radially outwards from a non-locking position where the locking arms 730 lie flush with the neighbouring surfaces of trigger element 700 and into a locking position where the lock surfaces 731 extend radially outwards from said neighbouring surfaces.

The needle shroud lock function further incorporates the power base 400. Power base 400 additionally includes two independent flexible arms 430 each extending in the distal direction from the power base. Each flexible arm is biased radially outwards so that a latch head 435 provided at the free distal end of the flexible arm assumes the position shown in FIG. 1b wherein the latch head 435 radially abuts the inner tubular surface of housing 300. Each latch head 435 comprises a radially outwards facing surface that is configured to cooperate by resiliently sliding against corresponding profiled axial tracks 736 of trigger element 700.

As shown in FIGS. 1b and 6a/6b, the deflectable arms 730 assume an unbiased non-locking radial position when the deflectable arms are not engaged by latch heads 435 of the two flexible arms 430. Each of the latch heads 435 of the two flexible arms 430 are configured to provide a radially outwards directed force on the corresponding deflectable arm 730 when the locking sleeve 700 is situated in the fired position, i.e. as shown in FIG. 4c. In this position each of the latch heads 435 grips behind a one-way latch protrusion 735 residing in the profiled axial track 736. Thus, when the trigger element 700 assumes the fired position the trigger element is arrested in the fired position and is prevented from moving distally again by latching engagement between latch heads 435 and one-way latch protrusions 735. For comparison the state shown in FIG. 3d provides a view of the device 10 just prior to the trigger element in the firing position where the latch heads 435 are positioned proximally relative to the one-way latch protrusions 735 and thus latch heads 435 are not yet latched. In both the firing position and in the fired position the flexible arms 430 of the power base 400 provides a radially outwards biased force onto the corresponding deflectable arm 730 of the trigger element. As the legs of the needle shroud 600 are positioned between the deflectable arms 730 and the housing 300 the deflectable arms 730 are still prevented from moving radially outwards into their locking position.

FIGS. 4a and 4b provides views of the device 10 in a state where the trigger element 700 is in the fired position, and the plunger 500 has already been caused to expel the dose of the syringe by plunging forward the piston 120 of syringe 100. The series of progression clicks have been generated during expelling and the piston has bottomed out in syringe barrel 100. The latch heads 435 of power base 400 grips behind the one-way latch protrusions 735 and. Thus, trigger element 700 is arrested in the fired position.

When the device 10 is removed from the injection site S the needle shroud spring 650 forces the needle shroud 600 from the proximal collapsed position into the distal extended locked position. During this movement, the proximally facing abutment surfaces 611 of the legs of the needle shroud initially moves out of engagement with the distal abutment surfaces 721 of the trigger element 700. Continued distal movement makes the legs of the needle shroud slide along the trigger element 700 until the proximally facing abutment surfaces 611 axially align with the distally directed lock surfaces 731 of the deflectable arms 730. Due to the radially outwards biased force from the flexible arms 430 onto the cooperating deflectable arms 730, the deflectable arms 730 are forced to move radially outwards into their locking position. As a consequence, the distally directed lock surfaces 731 of the deflectable arms 730 enter into blocking position relative to the proximally facing abutment surfaces 611 of the legs of the needle shroud 600 and the needle shroud 600 is prevented from moving towards the proximally collapsed position after the device 10 has been triggered.

Returning now briefly to details which relate to the triggering procedure of injection device 10 wherein the needle shroud 600 and the trigger element is moved in the proximal direction relative to the housing 300. Due to the profiled nature of axial tracks 736 the latch heads 435 initially climb a steep portion of the profiled axial tracks 736 (climb meaning move in the radial direction). This creates an initially high force which has to be overcome by the user when pushing device 10 against an injection site S to make the needle shroud 600 move towards the proximal collapsed position.

When the trigger element 700 is moved from the distal extended position towards the proximal collapsed position the two flexible arms 430 and the corresponding profiled axial tracks 736 of the trigger element 700 provide resistance to movement of the trigger element 700 and thus also resistance to movement of the needle shroud 600. Upon applying the autoinjector 10 at an injection site, a high axial reaction force is initially created when the flexible arms 430 engage the proximal end portion of the profiled axial tracks 736. Thus, a high force exerted on the needle shroud 600 is required in order for the flexible arms 430 to climb the profiled axial tracks 736. As soon as the flexible arms 430 have climbed the profiled axial tracks 736, resulting in the flexible arms 430 have been deformed radially inwards, the flexible arms 430 travel and slide along an almost constant height track profile as the needle shroud 600 is pushed further proximally relative to housing 300. This action requires considerable less force to be applied on the needle shroud 600 than the initial high force. Hence the needle shroud displacement will occur in two stages, i.e. a first high force stage and a second low force stage.

It will be appreciated, that the force needed for proximally displacing the needle shroud will be largely independent from the force provided by the drive spring, but will rather be decided by the force of the needle shroud spring 650 and the force profile for the interaction between the flexible arms 430 and the profiled axial tracks 736. A further minor force which has to be overcome when pushing in the needle shroud 600 emanates from the flexible arms 330 of the housing cooperating with the inner tubular proximal rim 635 of the needle shroud 600, cf. the discussion mentioned above with respect to the removal of the protective cap/RNS.

As will be discussed further below, the above- mentioned firing position of trigger element 700, and the corresponding position of needle shroud 600, will be situated at the final part of the proximal needle shroud movement where the flexible arms 430 travel along the almost constant height profile of axial tracks 736. The high initial needle shroud force over a short distance assures that the needle shroud is fully displaced and the autoinjector is effectively triggered due to the inertia of the human motion.

Turning now to FIGS. 7 through 10, these figures show a first example autoinjector 10′ in accordance with a first aspect of the present invention. The overall structure of autoinjector 10′ generally corresponds to the autoinjector 10 described above but in an embodiment, which has been modified enabling registering of triggering of the injection device by means of an electronic module 80′.

Relative to the embodiment 10 described above, the housing 300 of autoinjector 10′ has been prolonged slightly to be able to additionally accommodate an electronic module 80′ within the extreme proximal portion of the tubular shell of housing 300. Further, the power base 400 has been modified so as not to be fixedly attached relative to housing 300 but rather be movable axially within housing 300. In the shown embodiment the power base 400 is configured to be movable between a first pre-firing position (shown in FIG. 7) and into a second fired position (shown in FIG. 8).

The expelling assembly of the autoinjector 10′ is provided as a pre-assembled unit which in the following will be referred to as “power unit” 15′. Referring to FIG. 9, the power unit 15′ is formed as an assembly of components which in the shown embodiment is made up by the following components: plunger 500, drive spring 550, power base 400 and trigger element 700. Once assembled to form power unit 15′ the power unit serves as a pre-assembled and pre-energized drive assembly to be inserted into housing 300 as part of assembling the pre-filled syringe 100 with the remaining components of injection device 10′. The power unit 15′, even though it holds the drive spring 550 in a compressed state, forms a stable assembly where the trigger element 700 maintains the retaining elements 410/415 of the power base 400 securely engaged with the corresponding retaining surfaces of the plunger 500 during storage and handling of the energized power unit. In the shown embodiment the power base 400 is formed with a proximal base portion having radially outwards facing surfaces which are configured to slidably engage with mating surfaces provided on the radially inwards facing surface portions of the housing shell. On the proximal facing end face of power base 400 a switch actuator 450 is located. In the assembled state of the autoinjector 10′, the switch actuator 450 points towards the electronic module 80′ enabling the switch actuator to cooperate with a switch located on the distal facing portion of electronic module 80′.

FIG. 10 is a phantom representation of main components of the electronic module 80′ in which, to improve clarity, the electronic module housing 800 and parts relating to a dome 850 of a dome switch has been omitted from view (cf. FIGS. 7 and 8). The shown embodiment includes a flexible PCB 810 which is maintained in a multi-layered sandwich configuration by means of cooperating surfaces of electronic module housing 800. Flexible PCB 810 includes electronic circuitry and connects to either sides of a button cell battery 860. Furthermore, a processor 820 and other non-referenced electronic components are shown mounted onto PCB 810. Shown in FIG. 10 is further an area designated for an antenna 830 enabling wireless communication with an external electronic device, such as a Smartphone or other similar computing device. On the side of flexible PCB 810 which in the assembled state of the autoinjector 10′ faces the switch actuator 450 of the power base 400, electrode areas 851 are further shown. These form part of a dome switch together with the dome 850 cf. FIGS. 7 and 8. The electronics of electronic module 80′ also include various components, such as a crystal, a memory and wireless communication means coupled to the antenna and the processor.

As noted above FIG. 7 shows the proximal portion of the autoinjector 10′ in a state prior to triggering, i.e. in the state prior to the needle shroud has moved the trigger element 700 in the proximal direction. In this state the trigger element 700 cooperates with the retaining elements 410/415 to maintain the drive spring 550 in the tensed state between power base 400 and plunger 500, i.e. where the spring is compressed axially before administration. The power base 400 is mounted axially movable inside housing shell 300 between two end positions. In the shown embodiment, in the state shown in FIG. 7, the power base 400 is prevented from moving distally by a not shown retaining mechanism provided between power base 400 and housing 300. In the shown embodiment, power base is further prevented from moving proximally into its second fired position by cooperating with the dome 850 of the dome switch. Dome 850 is so configured that the dome will not axially collapse, relative to a non-collapsed state, unless a predefined collapsing force acts upon the dome.

In other embodiments, a snap mechanism may alternatively, or additionally, be provided between the power base 400 and the housing, wherein the snap-connection only releases for power base proximal movement when a proximal directed force of predefined magnitude acts upon the power base. Hence, the release of the snap mechanism is generally prevented unless the drive spring 550 is allowed to expand. Also, although not shown, the needle shroud 600 and/or the trigger element 700 may cooperate with not shown stops in the housing for limiting proximal movement of trigger element 700 and needle shroud 600 when the needle shroud is pressed proximally for performing an injection.

Referring to FIG. 8, for performing an injection, and cf. to the procedure shown in the series of views in FIGS. 1a through 4b, the autoinjector 10′ is triggered by pressing the distal end of the autoinjector towards the injection site. Hence, the needle shroud 600 is pushed proximally while carrying with it the trigger element 700 until the retaining elements 410/415 become released from the plunger 500. As this occurs, the drive spring 550 expands. Initially, the released force provided by the proximal end of the drive spring 550 urges the power base 400 proximally relative to the position shown in FIG. 7 while pressing dome 850 into a collapsed state. At the same time the distal end of the drive spring 550 acts on the plunger 500 which drives piston 120 distally for expelling the entire contents of the syringe 100. The movement of the power base 400 from the first non-fired position to the second fired position is associated with an audible click sound indicating initiation of the expelling procedure.

The electronic module 80′ is configured to store a time parameter relating to the time that autoinjector is triggered. Due to the dome 850 being collapsed upon triggering, the dome switch 850/851 is operated which will be registered by the processor 820 of the electronic module 80′. In the shown embodiment, this initialises a timer which is operated so that a counter starts counting. In the shown embodiment, the parameter stored in the counter represents elapsed time since initialisation, and retrospectively, the exact time of triggering may be calculated. In the shown embodiment, the electronic module comprises wireless communication means so that the time parameter stored in a memory, optionally with other data, is transferred to an external computing device, such as a smartphone device. The external computing device may then simply calculate the exact time of triggering, i.e. the real time value of triggering, by using the stored time parameter along with the real time wherein data has been transferred. In other embodiments, upon triggering of the device, the time stamp associated with the time of triggering, i.e. a real time value, is stored in a memory for later retrieval.

In an exemplary embodiment the wireless communication means is formed by NFC, Bluetooth®, Bluetooth® Low Energy (BLE) or similar means. The electronic module may be configured to store different types of data along with the time parameter, such as medicament type of the drug accommodated in the autoinjector, dose size, a unique serial number for the autoinjector, etc. Also, the electronic module may include a specific network address, such as a hyperlink, enabling the external computing device to download an app and/or configure itself for use and in accordance with the particular type of drug accommodated in the autoinjector.

In particular embodiments, the dome 850 of the electronic module 80′ provides a resilient biasing force on the power base 400 urging the power base distally away from the second fired position when the trigger element 700 assumes the pre-firing state. This ensures that the power base 400 is situated at the first pre-firing position as long as the device has not been triggered. Should the autoinjector 10′ become exerted to an impact, for example due to a user dropping the autoinjector, there is a risk that power base 400 will move into the second fired position although the autoinjector has not been deliberately triggered. After the impact, the dome 850 will push the power base back to the first pre-firing position.

The processor 820 of the electronic module may be configured to operate a timing means, such as a timer, to monitor the duration that the power base 400 assumes in the second fired position. The processor 820 may be so configured that the processor exclusively registers triggering of the injection device if said duration is longer than a pre-defined time limit, such as a time limit in the order of 1 second. Hence, If the power base 400 is returned to the first pre-firing position within less than 1 second, the electronic module 80′ will disregard the signal recorded by means of the dome switch 850/851, and the electronic module 80′ will be ready again to register a true deliberate triggering of the autoinjector 10′. Only upon an intentional controlled triggering of the autoinjector 10′ so that the drive spring 550 is allowed to expand, continued force of the drive spring 550 onto the power base 400, even after full expelling of the drug contained in the syringe 100, will result in the power base assuming the second fired position for a prolonged time. In the shown embodiment, the drive spring 550 provides a force much higher than the bias urged by dome 850, even when the drive spring assumes the state shown in FIG. 8, i.e. after expelling of the full dose. In this state, the force provided by drive spring 550 onto power base 400 will be of magnitude Fₘᵢₙ. The resilient biasing force provided by the dome switch 850/851 onto the power base 400 is thus smaller than the force Fₘᵢₙ, in typical embodiments of considerable smaller magnitude than Fₘᵢₙ.

Due to the simplicity of the electronic module, and the simplicity of the power unit, different versions of an autoinjector may be formed still utilizing the same power unit, and optionally, also the same housing 300. A first version may thus include the electronic module 80′ which thus provides an autoinjector that is electronically enabled, and a second version may include a cap that replaces the electronic module 80′, and wherein the cap includes no electronic components. The second version may not be electronically enabled, and thus less expensive to provide. In further embodiments, the power base may be formed so that it will mount axially fixed to the housing of a first non-electronic version of an autoinjector, and wherein the same power base is formed to be axially floating when inserted into the housing of an electronic version of the autoinjector. Hence, for the non-electronic version of the autoinjector, the power base may form a proximal end-cap member for capping off the proximal portion of the housing of the autoinjector. Thus, the need for a dedicated end-cap for the non-electronic version of the autoinjector is dispensed with.

In accordance with the above, an inexpensive production setup is provided wherein only a minimum number of variants of components and/or assemblies are needed for creating different versions of the autoinjector.

In accordance with a third aspect of the present invention FIGS. 11 through 13 depict details relating to a second example autoinjector 10″. The overall structure of autoinjector 10″ generally corresponds to the autoinjector 10 described above but with modifications enabling registering of operation of the injection device by means of an electronic circuitry arranged as part of a modified power base, i.e. as power base assembly 400″. Whereas the embodiments 10 and 10′ described above includes a plunger which is solid and wherein the drive spring is provided as a compression spring encircling the plunger, the plunger 500 of autoinjector 10″ is provided as a hollow elongated plunger wherein a compression spring 550 is arranged internally along the axis of the plunger. In other embodiments of autoinjector 10″, a solid plunger as shown in connection with devices 10 or 10′ may be used as an alternative.

In the embodiment shown in FIG. 11, the power base assembly 400″ includes a main part 401, a mounting part 405, and an electronic dose sensing circuitry, the latter including a carrier foil 840, two sensor parts, a processor 820, a battery 860, and other electronic components such as antenna and communication circuitry. The main part 401 includes a central axially extending pin 402 that serves as a spring guide that prevents the compressed drive spring 550 from buckling.

FIG. 13 shows an external perspective view of the main part 401 and the components of the dose sensing circuitry, but with the mounting part 405 omitted for clarity. In the power base assembly 400″ the dose sensing circuitry is arranged between the main part 401 and the mounting part 405 so that the mounting part 405 clamps the battery 860, carrier foil 840 and the two sensor parts when the mounting part 405 is attached to main part 401.

In the shown embodiment, the electronic dose sensing circuitry is provided on carrier foil 840 which is formed as a flexible sheet, wherein wiring and sensor circuitry including strain sensitive piezoelectric material 845 is printed or otherwise disposed onto the carrier foil. In the shown embodiment, the carrier foil 840 is provided as a thin deflectable sheet formed by a polymeric material, such as PET. The carrier foil 840 includes a main portion arranged transversely to the axis, wherein the main portion carries the processor 820 and a first electrode for coupling to a first electrode of the battery 860. The carrier foil further includes a first narrow section which couples to the main portion of the carrier foil. The first narrow section is folded around the battery 860 and includes a second electrode for coupling to a second electrode of the battery. Carrier foil 840 further includes second and third narrow sections that also couples to the main portion of the carrier foil. The second and third narrow sections are folded from opposite portions of the main portion to extend axially in the distal direction so that each of the second and third narrow sections lies flat against a portion of a respective one of the two resilient arms of the retaining elements 410/415.

Each of the second and third narrow sections of carrier foil 840, in combination with piezoelectric material 845 disposed at the free end of the narrow sections, forms a deflectable transducer which is configured to generate a signal when the deflectable transducer is deflected radially.

The mounting part 405 includes and end surface which forms an end-cap for the device 10″ when the power base assembly 400″ has been received and mounted relative to the housing 300 of the device 10″. The mounting part 405 further includes two axially extending sections each having a mounting protrusion 406 that is received within a respective mounting slot (non-referenced) formed in the proximal end portion of housing 300. In the shown embodiment, the power base assembly 400″ is mounted inside housing 300 so that power base 400″ is neither able to slide axially nor to move rotationally. Each of the two axially extending sections of mounting part 405 ends in a free resilient arm 403 that serves to provide radial pressure onto the respective second and third narrow section of the carrier foil 840, i.e. at a base portion of the deflectable transducer. Hence, each of the two deflectable transducers will generally follow radial movement of their respective resilient arm of the retaining elements 410/415. In accordance herewith, when the resilient arms of the retaining elements 410/415 deflect radially during the expelling movement of the plunger 500, the respective resilient transducer will generate a signal which is generally proportional to the amount of radial deflection of the resilient arm of the retaining elements 410/415.

In the shown embodiment, each resilient arm of the retaining elements 410/415 defines a support part 411 arranged to support a base part of the deflectable transducer. The support part 411 is arranged axially at the location where the free resilient arm 403 of the mounting part 405 meets the deflectable transducer. An undercut section 412 extends from the support part 411 and distally to a protrusion 413 arranged to support a free end of the deflectable transducer. Hence, between the support part 411 and the protrusion 413 the deflectable transducer is arranged non-supported by material portions of the resilient arm 410. A large portion of the piezoelectric material 845 may be disposed at the non-supported part of the deflectable transducer, in particular in the vicinity of the free resilient arm 403 of the mounting part 405. Compared to embodiments wherein the deflectable transducer is supported along the entire length of the transducer, the non-supported configuration ensures that the strain experienced by the deflectable transducer will be larger. Hence, a larger signal may be generated, facilitating improved detection of movement of the resilient arms of the retaining elements 410/415.

FIG. 12 shows the autoinjection device 10″ after the device has been triggered, i.e. by movement of the trigger element 700 from the pre-firing position to the firing position. In this state, the enlarged blocking heads 415 of the retaining elements are free to move radially, and will move radially outwards and inwards as the plunger 500 is thrust forward by the drive spring 550. In FIG. 12, the arms of retaining elements 410/415 have been moved radially outwards four times in total, initially by cooperating with the pair of stepped blocking geometries 515, and subsequently by cooperating with the first three plunger protrusions 525. Each separate movement of the retaining elements 410/415 is associated with a click sound generated by the device 10″, and a corresponding signal is recorded by the deflectable transducers and the associated processor 820. In the shown embodiment, eight click signals signify expelling of the full dose of drug from the syringe 100, and the plunger 500 will move distally for the remaining dose to be expelled, during which the last four clicks and transducer signals are generated as the retaining elements 410/415 move radially inwards and outwards.

In the shown embodiment, when eight clicks have been generated by each of the deflectable transducers, this initialises a timer which is operated so that a counter starts counting. In the shown embodiment, the parameter stored in the counter represents elapsed time since initialisation, and retrospectively, the exact time of triggering may be calculated. In the shown embodiment, the electronic module comprises wireless communication means so that the time parameter stored in a memory, optionally with other data, such as drug type, and/or a serial number for the device, is transferred to an external computing device, such as a smartphone device.

In other embodiments, the processor 820 may be configured to provide for real time monitoring of the expelling sequence. For example, each signal represented by a click may be transferred by the wireless communication means to provide a presentation on an external device so that the user may be guided during dose injection, such as by presenting a dedicated signal when the End of Dose condition has ensued, i.e. after eight dosing clicks in total. Alternatively, the autoinjection device 10″ may incorporate User Interface electronics that are configured to issue a dedicated End of Dose signal when eight clicks have been recorded, signifying expelling of the total dose. If it is deemed necessary to have the needle inserted at the injection site for a short time subsequent to End of Dose, such as for a few seconds subsequent to End of Dose, the End of Dose signal may be delayed relative to the final click to signify the desired end of a needle insertion resting time.

In the embodiment shown in FIG. 11, the two deflectable transducers are arranged in a symmetrical configuration wherein the two transducers face each other around the axis of symmetry. By this arrangement, unwanted signals from the deflectable transducers, such as signals generated due to an impact if the device is being unintentionally dropped on a hard surface, can be filtered out.

Some preferred embodiments have been shown in the foregoing, but it should be stressed that the invention is not limited to these, but may be embodied in other ways within the subject matter defined in the following claims.

1. An autoinjection device for expelling a dose of drug, the autoinjection device comprising:
a housing having a proximal end (P) and a distal end (D),
a drug container comprising a container barrel extending along an axis, a distal outlet connectable or connected to an injection needle, and a piston that is sealingly and slideably arranged inside the container barrel,
a power unit configured for driving the piston distally along the axis to expel a drug contained in the drug container the power unit comprising:
a plunger adapted for cooperation with the piston to drive the piston distally along a central axis, the plunger comprising a retaining geometry,
a drive spring arranged in a tensed state wherein a first end portion of the drive spring acts on the plunger with a force biasing the plunger distally, and
a power base operably coupled to the drive spring and the plunger, the power base grounding a second end portion of the drive spring, wherein, in a pre-firing state, a retaining element of the power base releasably engages the retaining geometry of the plunger to retain the plunger against the force of the drive spring,
a user operable trigger element cooperating with the retaining element and shiftable from a pre-firing state wherein the trigger element cooperates with the retaining element to maintain retaining engagement with the retaining geometry of the plunger, and into a firing state wherein said retaining engagement is released, wherein the power base is movably arranged in the housing and configured to move proximally from a first pre-firing position into a second fired position upon release of the retaining engagement, and
an electronic module arranged relative to the housing, the electronic module comprising a sensor configured to sense the shift of position of the power base as it moves from the first pre-firing position into the second fired position.

2. The autoinjection device as defined in claim 1, wherein, when the trigger element assumes the pre-firing state, the power unit is arranged axially floating relative to the housing.

3. The autoinjection device as defined in claim 1, wherein the retaining element is unitarily formed with the power base.

4. The autoinjection device as defined in claim 1, wherein the retaining element defines an arm extending from a base section of the power base towards a plunger engagement portion, wherein the arm is radially resilient to allow the plunger engagement portion to become radially shifted from an engagement position where the retaining engagement is maintained and into a release position where the retaining engagement is released.

5. The autoinjection device as defined in claim 1, wherein the power base and the housing comprises cooperating snap geometries for releasably retaining the power base in the first pre-firing position, said cooperating snap geometries being configured to release due to the force of the drive spring upon release of the retaining engagement.

6. The autoinjection device as defined in claim 1, and further comprising a biasing structure providing a resilient biasing force on the power base urging the power base distally away from the second fired position when the trigger element assumes the pre-firing state.

7. The autoinjection device as defined in claim 1, wherein the sensor of the electronic module comprises a switch that senses the shift of position of the power base as it moves from the first pre-firing position into the second fired position.

8. The autoinjection device as defined in claim 7, wherein the switch comprises a dome switch having a dome positioned for engagement, and being acted upon, by the power base.

9. The autoinjection device as defined in claim 7, wherein the power base defines a switch actuator configured to actuate the switch of the electronic module.

10. The autoinjection device as defined in claim 1, wherein the electronic module is provided as a self-contained electronics assembly, the electronic module being coupled to or received within the proximal end of the housing.

11. The autoinjection device as defined in claim 1, wherein the electronic module comprises an energy source and a processor coupled to the energy source and the sensor, the processor being configured to register triggering of the injection device by structure of the sensor sensing the shift of position of the power base as it moves from the first pre-firing position into the second fired position.

12. The autoinjection device as defined in claim 11, wherein the electronic module comprises timing structure, and wherein the processor is configured to operate the timing structure to monitor the duration that the power base assumes in the second fired position, and wherein registering triggering of the autoinjection device is made only if said duration is longer than a pre-defined time limit.

13. The autoinjection device as defined in claim 11, wherein the processor is configured to register time elapsed since registering triggering of the injection device.

14. The autoinjection device as defined in claim 1, wherein the electronic module comprises a wireless communication interface configured to communicate with an external electronic device.

15. The autoinjection device as defined in claim 1, wherein the power unit further comprises said user operable trigger element.
